(19) United States
(12) Patent Application Publication
(10) Pub. No.: US 2007/0014845 A1
(43) Pub. Date: Jan. 18, 2007

(54) Liposomal delivery vehicle for hydrophobic drugs

(76) Inventors: Yuanpeng Zhang, Cupertino, CA (US); Bing Luo, Fremont, CA (US); Laxmi Iyer, Milpitas, CA (US)

Correspondence Address: PERKINS COIE LLP, P.O. BOX 2168, MENLO PARK, CA, 94026, US

(21) Appl. No.: 11/479,620

(22) Filed: Jun. 30, 2006

Related U.S. Application Data
(60) Provisional application No. 60/695,961, filed on Jul. 1, 2005.

(51) Int. Cl. A61K 9/127; A61K 31/724

(52) U.S. Cl. 424/450; 514/58

(57) ABSTRACT

A liposome composition having a high drug concentration of a hydrophobic drug and capable of retaining the drug in entrapped form is described. The liposomes are comprised of high phase transition lipid and a lipopolymer, which together permit retention of a high concentration of a drug/cyclodextrin complex that achieves a high drug load that is retained even in the presence of a transmembrane osmotic gradient caused by the cyclodextrin.

### CROSS-REFERENCE TO RELATED APPLICATIONS

This application claims the benefit of U.S. Provisional Application No. 60/695,961, filed Jul. 1, 2005, incorporated herein by reference in its entirety.

### TECHNICAL FIELD

The subject matter described herein relates to a liposome composition containing a hydrophobic drug.

### BACKGROUND

Liposome delivery systems have been proposed as carriers for a variety of compounds, including pharmacologically active compounds, diagnostic agents, and cosmetics. Liposomes typically have one or more lipid bilayers enclosing one or more aqueous internal compartments, where the compound of interest is entrapped in either the aqueous internal spaces, in the lipid bilayer(s), or both, depending on the nature of the compound. Water soluble compounds are readily entrapped in the aqueous internal space(s), and a sufficient quantity, or load, of water soluble compounds can usually be achieved to arrive at a meaningful delivery system. Compounds that are poorly water soluble or hydrophobic compounds are not well suited for incorporation into the aqueous internal space(s). Instead, poorly water soluble compounds tend to be incorporated into the lipid bilayer(s), which has certain disadvantages. First, the presence of the compound in the lipid bilayer(s) can destabilize the liposome structure. Second, the quantity of compound that can be incorporated into the lipid bilayer(s) is limited.

Thus, there remains a need for a plasma stable liposome delivery system having a long blood circulation lifetime and capable of carrying a hydrophobic compound in a therapeutically meaningful amount.

### SUMMARY

Accordingly, in one aspect, a liposome composition comprised of liposomes having a vesicle-forming lipid exhibiting a phase transition above about 40° C., preferably above 50° C., and having between about 1-20 mole percent of a lipid derivatized with a hydrophilic polymer, is contemplated. Entrapped within an interior region of each liposome is a complex comprised of a hydrophobic drug and a cyclodextrin compound, the cyclodextrin being present in a concentration above about 100 mg/mL, preferably above about 200 mg/mL, more preferably above about 300 mg/mL, and still more preferably above about 400 mg/mL.

In another aspect, a process for preparing liposomes is provided. The process comprises providing a lipid mixture comprised of (i) of a vesicle-forming lipid having a phase transition above about 40° C. and (ii) between about 1-20 mole percent of a lipid derivatized with a hydrophilic polymer; combining the lipid mixture with a drug-cyclodextrin solution to form liposomes having a concentration of cyclodextrin of greater than about 200 mg/mL, more preferably of greater than about 400 mg/mL; and processing the liposomes to obtain a desired particle size.

In addition to the exemplary aspects and embodiments described above, further aspects and embodiments will become apparent by reference to the drawings and by study of the following descriptions.

### BRIEF DESCRIPTION OF THE DRAWINGS

FIG. 1 shows the structure of an exemplary hydrophobic drug identified herein as RWJ-416457.

FIG. 2 shows plasma concentration, in μg/mL, of the drug RWJ-416457 as a function of time, in hours, after administration of liposomes containing an inclusion complex of the drug to four dogs, the time points for each animal represented by a diamond, square, triangle or x symbol.

### DETAILED DESCRIPTION

I. Liposome Composition and Method of Preparation

In one aspect, a liposome composition having a poorly water soluble compound entrapped therein in the form of an inclusion complex with a complexation reagent, such as cyclodextrin, is provided. As used herein, the terms “poorly water soluble compound” and “hydrophobic compound” are used interchangeably to intend compounds that are sparingly soluble in water, as evidenced by a room temperature water solubility of less than about 100 μg/mL, and in some cases of less than about 50 μg/mL. Exemplary compounds and a room temperature water solubility value for an exemplary compound are provided below. It will be appreciated that the room temperature water solubility for any given compound can be easily determined using readily available chemistry techniques and tools, such as high performance liquid chromatography or spectrophotometry. The liposomes are comprised of a vesicle-forming lipid having a relatively high phase transition temperature, i.e., a saturated or rigid lipid, as will be further described below. The liposomes also include a lipid derivatized with a hydrophilic polymer, such as polyethylene glycol. Due to the presence of the cyclodextrin inside the liposomal interior, the liposomes exhibit a transmembrane (i.e., trans lipid bilayer membrane) osmotic gradient, yet are able to retain the drug, as will be demonstrated below.

The liposomes in the composition are composed primarily of one or more vesicle-forming lipids. Such a vesicle-forming lipid is one which can form spontaneously into bilayer vesicles in water, as exemplified by the phospholipids, with its hydrophobic moiety in contact with the interior, hydrophobic region of the bilayer membrane, and its head group moiety oriented toward the exterior, polar surface of the membrane. Lipids capable of stable incorporation into lipid bilayers, such as cholesterol and its various analogs, can also be used in the liposomes, as further illustrated below. The vesicle-forming lipids are preferably lipids having two hydrocarbon chains, typically acyl chains, and a head group, either polar or nonpolar. There are a variety of synthetic vesicle-forming lipids and naturally-occurring vesicle-forming lipids, including the phospholipids, such as phosphatidylcholine, fully or partially hydrogenated soy phosphatidylcholine, phosphatidylethanolamine, phosphatidic acid, phosphatidylinositol, phosphatidylglycerol and sphingomyelin, where the two hydrocarbon chains are typically between about 14-22 carbon atoms in length, and have varying degrees of unsaturation. The above-described lipids and phospholipids whose acyl chains have varying degrees of saturation can be obtained commercially or prepared according to published methods.

In a preferred embodiment, the liposomes are prepared from a lipid having a relatively high phase transition temperature to achieve a more rigid lipid bilayer, or a gel state (solid-ordered) phase bilayer. Relatively rigid lipids, e.g., a lipid having a relatively high phase transition temperature of greater than about 40° C., preferably greater than about 45° C., more preferably of greater than about 50° C., and still more preferably of greater than about 55° C., are described in the art and the phase transition temperatures of lipids are tabulated in various sources, such as Lipidat database and in Szoka, F. et al., Ann. Rev. Biophys. Bioeng., 9:467 (1980). Exemplary rigid lipids include distearoyl phosphatidylcholine (DSPC), which has a phase transition temperature of about 55° C., hydrogenated soy phosphatidylcholine (HSPC), which has a phase transition temperature of about 55° C.; distearoyl-phosphatidylglycerol (DSPG), which has a phase transition temperature of about 55° C.; dipalmitoylphosphatidylglycerol (DPPG), which has a phase transition temperature of about 41° C.; dipalmitoyl phosphatidic acid (DPPA), which has a phase transition temperature of about 58-67° C.; dipalmitoyl phosphatidylethanolamine (DPPE), which has a phase transition temperature of about 60° C.

Other lipid components, such as cholesterol, are also known to contribute to membrane rigidity in lipid bilayer structures by transforming the bilayer to be in a liquid ordered phase. Thus, an embodiment includes addition of cholesterol and/or a cholesterol derivative to the liposomes.

The liposomes also include a lipopolymer, i.e., a lipid covalently attached to a hydrophilic polymer. Lipopolymers, in particular mPEG-DSPE conjugates, have been used extensively in various liposomal delivery systems (Woodle, M. C. in POLY(ETHYLENE GLYCOL) CHEMISTRY AND BIOLOGICAL APPLICATIONS, J. M. Harris and S. Zalipsky, Eds., ACS Symp. Series 680, pp. 60-81, American Chemical Soc., Washington, DC. (1997)). As has been described, for example in U.S. Pat. No. 5,013,556, including such a polymer-derivatized lipid in the liposome composition forms a surface coating of hydrophilic polymer chains around the liposome. The surface coating of hydrophilic polymer chains is effective to increase the in vivo blood circulation lifetime of the liposomes when compared to liposomes lacking such a coating. Polymer-derivatized lipids comprised of methoxy(polyethylene glycol) (mPEG) and a phosphatidylethanolamine (e.g., dimyristoyl phosphatidylethanolamine, dipalmitoyl phosphatidylethanolamine, distearoyl phosphatidylethanolamine (DSPE), or dioleoyl phosphatidylethanolamine) can be obtained from Avanti Polar Lipids, Inc. (Alabaster, Ala.) at various mPEG molecular weights (350, 550, 750, 1000, 2000, 3000, 5000 Daltons). Lipopolymers of mPEG-ceramide can also be purchased from Avanti Polar Lipids, Inc. Preparation of lipid-polymer conjugates is also described in the literature, see U.S. Pat. Nos. 5,631,018, 6,586,001, and 5,013,556 (all incorporated by reference); Zalipsky, S., et al., Bioconjugate Chem. 8:111 (1997); Zalipsky, S., et al., Meth. Enzymol. 387:50, (2004). These lipopolymers can be prepared as well-defined, homogeneous materials of high purity, with minimal molecular weight dispersity (Zalipsky, S., et al., Bioconjugate Chem. 8:111, (1997); Wong, J., et al., Science 275:820, (1997)). The lipopolymer can also be a “neutral” lipopolymer, such as a polymer-distearoyl conjugate, as described in U.S. Pat. No. 6,586,001, incorporated by reference herein.

When a lipid-polymer conjugate is included in the liposomes, typically between 1-20 mole percent of the lipid-polymer conjugate is incorporated into the total lipid mixture (see, for example, U.S. Pat. No. 5,013,556). In one embodiment, between 2.5-15 mole percent of the lipid-polymer conjugate is included in the lipid mixture for liposome preparation.

The liposomes can additionally include a lipopolymer modified to include a ligand, forming a lipid-polymer-ligand conjugate, also referred to as a ‘lipopolymer-ligand conjugate’. The ligand can be a therapeutic molecule, such as a drug or a biological molecule having activity in vivo, a diagnostic molecule, such as a contrast agent or a biological molecule, or a targeting molecule having binding affinity for a binding partner, preferably a binding partner on the surface of a cell. A preferred ligand has binding affinity for the surface of a cell and facilitates entry of the liposome into the cytoplasm of a cell via internalization. A ligand present in liposomes that include such a lipopolymer-ligand is oriented outwardly from the liposome surface, and therefore available for interaction with its cognate receptor.

Methods for attaching ligands to lipopolymers are known, where the polymer can be functionalized for subsequent reaction with a selected ligand. (U.S. Pat. No. 6,180,134; Zalipsky, S. et al., FEBS Lett. 353:71 (1994); Zalipsky, S. et. al., Bioconjugate Chem. 4:296 (1993); Zalipsky, S. et al., J. Control. Rel. 39:153 (1996); Zalipsky, S. et al., Bioconjugate Chem. 8(2):111(1997); Zalipsky, S. et al., Meth. Enzymol. 387:50 (2004)). Functionalized polymer-lipid conjugates can also be obtained commercially, such as end-functionalized PEG-lipid conjugates (Avanti Polar Lipids, Inc.). The linkage between the ligand and the polymer can be a stable covalent linkage or a releasable linkage that is cleaved in response to a stimulus, such as a change in pH or presence of a reducing agent.

The ligand can be a molecule that has binding affinity for a cell receptor or for a pathogen circulating in the blood. The ligand can also be a therapeutic or diagnostic molecule, in particular molecules that when administered in free form have a short blood circulation lifetime. In one embodiment, the ligand is a biological ligand, and preferably is one having binding affinity for a cell receptor. Exemplary biological ligands are molecules having binding affinity to receptors for CD4, folate, insulin, LDL, vitamins, transferrin, asialoglycoprotein, selectins, such as E, L, and P selectins, Flk-1,2, FGF, EGF, integrins, in particular, α₄β₁αᵥβ₃, αᵥβ₁αᵥβ₅, αᵥβ₆ integrins, HER2, and others. Preferred ligands include proteins and peptides, including antibodies and antibody fragments, such as F(ab′)₂, F(ab)₂, Fab′, Fab, Fv (fragments consisting of the variable regions of the heavy and light chains), and scFv (recombinant single chain polypeptide molecules in which light and heavy variable regions are connected by a peptide linker), and the like. The ligand can also be a small molecule peptidomimetic. It will be appreciated that a cell surface receptor, or fragment thereof, can serve as the ligand. Other exemplary targeting ligands include, but are not limited to vitamin molecules (e.g., biotin, folate, cyanocobalamine), oligopeptides, oligosaccharides. Other exemplary ligands are presented in U.S. Pat. Nos. 6,214,388; 6,316,024; 6,056,973; 6,043,094, which are herein incorporated by reference.

The liposome composition also includes a cyclodextrin. Cyclodextrins are cyclic oligosaccharides of α-D-gluco-pyranose and can be formed by the catalytic cyclization of starch. Due to a lack of free rotation about the bonds connecting the glycopyranose units, cyclodextrins are toroidal or cone shaped, rather than cylindrical. The cyclodextrins have a relatively hydrophobic central cavity and a hydrophilic outer surface. The hydrophobic cage-like structure of cyclodextrins has the ability to entrap a variety of guest compounds to form host-guest complexes in the solid state and in solution. These complexes are often termed inclusion complexes and the guest compounds are released from the inclusion site.

Common cyclodextrins are α-, β-, and γ-cyclodextrin, which consist of six, seven, or eight glucopyranose units, respectively. Cyclodextrins containing nine, ten, eleven, twelve, and thirteen glucopyranose units are designated δ-, ε-, ξ-, η-, and θ-cyclodextrin, respectively. Characteristics of α-, β-, γ-, and δ-cyclodextrin are shown in Table 1.

TABLE 1
Cyclodextrin Characteristics

| | α-cyclodextrin | β-cyclodextrin | γ-cyclodextrin | δ-cyclodextrin |
|---|---|---|---|---|
| no. of glucopyranose units | 6 | 7 | 8 | 9 |
| molecular weight (Daltons) | 972 | 1135 | 1297 | 1459 |
| central cavity diameter (Å) | 4.7-5.3 | 6.0-6.5 | 7.5-8.3 | 10.3-11.2 |
| water solubility | 14.5 | 1.85 | 23.2 | 8.19 |
| (at 25° C., g/100 mL) | | | | |

Derivatives formed by reaction with the hydroxyl groups lining the upper and lower ridges of the toroid are readily prepared and offer a means of modifying the physicochemical properties of the parent cyclodextrins. The parent cyclodextrins, and in particular β-cyclodextrin, have limited aqueous solubility. Substitution of the hydroxyl groups, even with hydrophobic moieties such as methoxy and ethoxy moieties, typically increases solubility. The hydroxyl groups in the parent cyclodextrins can also be substituted with phosphate, sulfate, sulfoalkyl ether, carboxymethyl, and succinate groups. Since each cyclodextrin hydroxyl group differs in chemical reactivity, reaction with a modifying moiety usually produces an amorphous mixture of positional and optical isomers. The aggregate substitution that occurs is described by a term called the degree of substitution. For example, a 2-hydroxypropyl-β-cyclodextrin with a degree of substitution of five would be composed of a distribution of isomers of 2-hydroxypropyl-β-cyclodextrin in which the average number of hydroxypropyl groups per 2-hydroxypropyl-β-cyclodextrin molecule is five. Degree of substitution can be determined by mass spectrometry or nuclear magnetic resonance spectroscopy. These methods do not give information as to the exact location of the substituents (C1, C2, C3, etc.) or the distribution of the substituents on the cyclodextrin molecule (mono, di, tri, poly). Theoretically, the maximum degree of substitution is 18 for α-cyclodextrin, 21 for β-cyclodextrin, and 24 for δ-cyclodextrin, however, substituents with hydroxyl groups present the possibility for additional hydroxylalkylations. Properties of some common cyclodextrins are shown in Table 2.

TABLE 2

| | | | Solubility |
|---|---|---|---|
| | | | in water |
| Cyclodextrin | Substitution | MW | (mg/ml) |
| α-Cyclodextrin | — | 972 | 145 |
| β-Cyclodextrin | — | 1135 | 18.5 |
| 2-Hydroxypropyl-β-cyclodextrin | 0.65 | 1400 | >600 |
| Randomly Methylated β-cyclodextrin | 1.8 | 1312 | >500 |
| β-Cyclodextrin sulfobutyl | 0.9 | 2163 | >500 |
| ether sodium salt | | | |
| γ-Cyclodextrin | — | 1297 | 232 |
| 2-Hydroxypropyl-γ-cyclodextrin | 0.6 | 1576 | >500 |

The cyclodextrin used in the composition described herein is preferably one that has a room temperature water solubility of above 20 w/v percent and can be an α-, β-, or γ-cyclodextrin. In a preferred embodiment, a derivative of a cyclodextrin is selected, and derivatives such as hydroxypropyl, dimethyl, and trimethyl substituted cyclodextrins are contemplated, as are cyclodextrins linked with sugar molecules, sulfonated cyclodextrins, carboxylated cyclodextrins, and amino derivatives such as diethylamino cyclodextrins. In a preferred embodiment, the cyclodextrin is a β-cyclodextrin, and in a more preferred embodiment, the cyclodextrin is 2-hydroxypropyl-o-cyclodextrin or sulfobutyl ether-β-cyclodextrin (Captisol®). In yet another embodiment, the 2-hydroxypropyl-β-cyclodextrin has a degree of substitution between 2 and 8, more preferably between 4 and 8, most preferably between 5 and 8.

The liposomes also include a drug entrapped in the aqueous space(s) of the liposome, substantially in the form of an inclusion complex with the cyclodextrin. In a preferred embodiment, the drug is a hydrophobic compound, however it will be appreciated that the formulation can also be used for hydrophilic compounds. As noted above, a hydrophobic compound is one that has poor room temperature water solubility, typically a water solubility of less than about 500 μg/mL, more preferably less than about 100 μg/mL. The entrapped drug can be any desired compound, without limitation, either natural or synthetic. A therapeutic agent can be a pharmaceutical agent, including biologics such as proteins, peptides, and nucleotides, or a diagnostic agent, such as a contrast agent, including x-ray contrast agents.

The drug can be selected from a variety of known classes of drugs, including, for example, proteins, peptides, nucleotides, anti-obesity drugs, nutriceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. The drugs are commercially available and/or can be prepared by techniques known in the art.

Some preferred drugs include steroids, immunosuppressants, antihistamines, non-steroidal anti-asthamtics, non-steroidal anti-inflammatory agents, cyclooxygenase-2 inhibitors, cytotoxic agents, gene therapy agents, radiotherapy agents, and imaging agents. The entrapped therapeutic agent is, in one embodiment, a cytotoxic drug. Examples include an anthracycline antibiotic, a platinum compound, a topoisomerase 1 inhibitor, a vinca alkaloid, or an angiogenesis inhibitor.

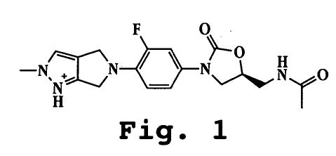

In another embodiment, the entrapped drug is an anti-microbial agent, and in particular is an antimicrobial compound effective to treat infections due to gram positive bacteria. More particularly, the drug is effective to treat multi-drug resistant gram positive bacterial infections, such as methicillin-resistant Staphylococcus aureus, a common nosocomial infection. Vancomycin resistant microorganisms are also becoming prevalent. Accordingly, a liposome formulation including an antimicrobial agent with activity against multi-drug resistant, e.g., methicillin, penicillin, and/or vancomycin, gram positive bacteria, is contemplated. Compounds belonging to the class of oxazolidinones are preferred, such as linezolid, 4-substituted 1,2,3-triazoles (Reck, F. et al., J. Med. Chem., 48(2):499-506 (2005)), and RWJ-416457, the structure of which is shown in FIG. 1. Various derivatives of oxazolidinones are also described in the art, such as phenyl derivatives disclosed in US 2004/0254162, incorporated by reference herein.

B. Preparation of Liposome Formulation

Various liposomal formulations were prepared to perform supporting and illustrative studies. In a first study, described in Example 1, the poorly water soluble (solubility is less than about 20 μg/mL at room temperature) oxazolidinone compound RWJ-416457 was added to a 40 w/v percent solution of hydroxypropyl-β-cyclodextrin or to a 20 w/v percent solution of sulfobutyl ether-β-cyclodextrin under various conditions of pH, temperature, and incubation time. The solubility of the drug in the cyclodextrin solutions was determined. The complete results are summarized in the table presented in Example 1. In brief, the antimicrobial oxazolidinone compound had a solubility of about 9.1 mg/mL in 40 w/v % hydroxypropyl-β-cyclodextrin at 45° C. and a solubility of about 7.6 mg/mL in 40 w/v % hydroxypropyl-β-cyclodextrin at room temperature. The drug had a solubility of about 4.5 mg/mL in 20 w/v % sulfobutylether-β-cyclodextrin at 45° C.

Accordingly, contemplated is a liposome composition comprising a cyclodextrin at a concentration of at least about 100 mg/mL, preferably of at least about 200 mg/mL, more preferably of at least about 300 mg/mL, and still more preferably of at least about 400 mg/mL.

Liposomes were prepared as described in Examples 1B-1C from various lipid mixtures and concentrations of cyclodextrin. In brief, and as summarized in Table 3, liposomes were prepared using a 50 w/v % solution of hydroxypropyl-beta-cyclodextrin (500 mg/mL, HPβCD) containing 13 mg/mL drug (formulation nos. 3, 4, 7, and 9-11), a 30% w/v % (300 mg/mL) solution of cyclodextrin containing 9.9 mg/mL of drug (formulation no. 5), or a 20% w/v % (200 mg/mL) solution of cyclodextrin containing 9.9 mg/mL of drug (formulation nos. 6 and 8). The 50 w/v % cyclodextrin/drug solution had an osmolality of about 720 mOsm/kg. The cyclodextrin/drug solutions were mixed with an ethanol solution of lipids having various compositions as follows (i) hydrogenated soy phosphatidylcholine (HSPC), (ii) HSPC and cholesterol, (iii) HSPC and methoxypoly(ethyleneglycoly)-distearolyphosphatidylethanolamine (mPEG-DSPE); or (iv) HSPC, cholesterol, and MPEG-DSPE. The mixture of the lipid solution and the cyclodextrin/drug solution was stirred to form liposomes, which were then subjected to extrusion through various pore-sized filters, followed by diafiltration to remove any unentrapped cyclodextrin/drug and ethanol (Example 1C-1D). Table 3 summarizes the liposome compositions, particle size and drug loading concentration (potency) for formulation nos. 1-11.

TABLE 3

| Formulation | Lipid | | Particle Size | Lipid | Drug | |
|---|---|---|---|---|---|---|
| No. | Components | | Post Extrusion | Conc. | Potency | Drug/lipid |
| (batch No.) | (mol/mol) | Hydration Medium¹ | (nm; 90°) | (mM)² | (mg/mL)² | g/mole |
| 1 | HSPC | saline/EtOH (10 v/v %) | 515 | ≈90 | n/d | n/d |
| 2 | HSPC/chol | saline/EtOH (10 v/v %) | 185 | ≈90 | n/d | n/d |
| | (55:45) | | | | | |
| 3 | HSPC | drug (12.95 mg/mL)/ | 348 | 158.24 | 0.18 | 1.1 |
| | | HPβCD (50 w/v %)/EtOH | | | | |
| | | (10 v/v %) | | | | |
| 4 | HSPC/chol | drug (12.95 mg/mL)/ | 214 | 74.4 | 0.08 | 1.1 |
| | (55:45) | HPβCD (50 w/v %)/EtOH | | | | |
| | | (10 v/v %) | | | | |
| 5 | HSPC/Chol | drug (9.9 mg/mL)/HPβCD | 217 | 146.57 | 0.22 | 1.5 |
| | (55:45) | (30 w/v %)/EtOH (10 v/v %) | | | | |
| 6 | HSPC/Chol | drug (4.8 mg/mL)/HPβCD | 220 | 80.2 | 0.17 | 2.1 |
| | (55:45) | (20 w/v %)/EtOH (10 v/v %) | | | | |
| 7 | HSPC/chol/mPEG- | drug (12.6 mg/mL)/HPβCD | 147 | 115.2 | 1.82 | 15.8 |
| | DSPE | (50 w/v %)/EtOH (10 v/v %) | | | | |
| | (52.5:45:2.5) | | | | | |
| 8 | HSPC/chol/mPEG- | Drug (5.1)/HPβCD (20 | 158.7 | 115.8 | 0.9 | 7.8 |
| | DSPE | w/v %)/EtOH (10 v/v %) | | | | |
| | (50:45:5) | | | | | |
| 9 | HSPC/chol/mPEG- | drug (13 mg/mL)/HPβCD | 182 | 114.76 | 1.19 | 10.4 |
| | DSPE | (50 w/v %)/EtOH (10 v/v %) | | | | |
| | (54:45:1) | | | | | |
| 10 | HSPC:chol:mPEG- | drug (13.0 mg/mL)/HPβCD | 115 | 166.6 | 1.79 | 10.7 |
| | DSPE | (50 w/v %)/EtOH (10 v/v %) | | | | |
| | (50:45:5) | | | | | |
| 11 | HSPC/mPEG- | drug (13.0 mg/mL)/HPβCD | 126 | 91.1 | 1.91 | 21.0 |
| | DSPE | (50 w/v %)/EtOH (10 v/v %) | | | | |
| | (95:5) | | | | | |

¹all hydration medium contained 10 mM NaCl and 15 mM histidine (pH 6.8-7.5)
²lipid concentration and drug potency were obtained post diafiltration against 1 wt % of NaCl, except for formulation nos. 6 and 8 for which diafiltration was against 0.6 wt % of NaCl solutions.

The data in Table 3 shows that liposomes comprising a lipopolymer, exemplified by mPEG-DSPE, have a significantly higher concentration of entrapped drug than liposomes lacking the lipopolymer. Formulation nos. 3 and 4, which were prepared from 500 mg/mL HPβCD and no lipopolymer, had very low drug loadings of 1.1 g/mole (as evidenced by the drug-to-lipid ratio in g/mole) regardless of whether cholesterol was included in the formulation. Formulation no. 5, which was prepared at a reduced HPβCD concentration of 300 mg/mL (30 w/v %) and no lipopolymer in the lipid composition, had a very low drug loading of 1.5 g/mole. Formulation no. 6, which was prepared at a reduced HPβCD concentration of 200 mg/mL (20 w/v %) and no lipopolymer in the lipid composition, had a very low drug loading of 2.1 g/mole. In formulation no. 6, the liposomes lacked a transmembrane gradient, since the internal and external phases were of approximately equal osmolalities (20 w/v % hydroxypropyl-β-cyclodextrin, osmolality of 198 mOsm/kg; 0.6% NaCl external medium, osmolaltiy of about 200 mOsm/kg). In comparison, formulation no. 8, which is identical to formulation no. 6 except for the presence of 5 mol% mPEG-DSPE in the lipid composition, had a drug loading of 7.8 g/mole. Thus, addition of a hydrophilic polymer to the lipid composition achieved a 3-4 fold increase in drug loading, at 20 w/v % cyclodextrin. As will be seen below, considerably higher improvements were achieved by the addition of a hydrophilic polymer to the lipid compositions when higher cyclodextrin concentrations were used in the hydration medium.

A comparison of formulation nos. 4, 10, and 11 also illustrate the unexpected findings achieved when a hydrophilic polymer is part of the lipid composition. Formulation nos. 4, 10, and 11 each have 50 w/v % hydroxypropyl-o-cyclodextrin and a 13 mg/mole drug concentration in the hydration medium. Formulation no. 4, which lacked a hydrophilic polymer in the lipid composition, had a drug load post-liposome formation of 1.1 g/mole. Formulation nos. 10 and 11, which both contained 5 mole percent of a lipopolymer (mPEG-DSPE) had drug loadings of 10 and 20 g/mole, respectively. Thus, addition of a lipopolymer to the liposome lipid bilayer resulted in a 10-20 fold increase in the amount of drug that could be loaded into the liposomes.

To further illustrate that the improved drug loading is due to the hydrophilic polymer, liposome formulations with reduced molar content of mPEG-DSPE were prepared. Specifically, liposome preparations with 2.5 mole percent and 1 mole percent were prepared, with 50 w/v % hydroxypropyl-β-cyclodextrin (formulation nos. 7 and 9). Formulation no. 7 comprised of HSPC/cholesterol/mPEG-DSPE (52.5:45:2.5) and formulation no. 9 comprised of HSPC/cholesteroVmPEG-DSPE (54:45:1) had drug loading of 15.8 g/mole and 10.4 g/mole, respectively, which are in the same range as for formulations containing 5 mol % mPEG-DSPE, but significantly higher than liposome formulations lacking the lipopolymer. Thus, in one embodiment, the liposomes include between about 1-10 mole percent of a lipopolymer, which permits formation of liposomes having a concentration of hydrophobic drug, in the form of a cyclodextrin/drug complex, of drug-to-lipid ratio of at least about 10 g/mole, more preferably of at least about 15 g/mole, and even more preferably of at least about 20 g/mole.

A 50 w/v % cyclodextrin solution has an osmolality of about 720 mOsm/kg, and liposomes prepared with a 50 w/v % cyclodextrin solution have a significant osmotic gradient across the lipid bilayer, the gradient depending on the osmolality of the solution in the external suspension medium. Typically, the external suspension medium is an isotonic saline, suitable for intravenous administration, such as 1% percent sodium chloride, which has an osmolality of about 350 mOsm/kg. Thus, a liposome composition comprising an internal solution of 50 w/v % hydroxypropyl-β-cyclodextrin and an external suspension medium of isotonic saline has a transmembrane osmotic gradient of at least 350 mOsm/kg. The inside (inner liposome) osmolality is about two-fold higher than the outside (external suspension medium) osmolality. Studies were done to determine if the two-fold higher inside/lower outside transmembrane osmotic gradient resulted in drug leakage from the liposomes. In a first study, described in Example 2, liposomes having an internal solution of 50 w/v % hydroxypropyl-β-cyclodextrin with an entrapped drug were mixed with fetal bovine serum or with 2% sodium chloride solution. Fetal bovine serum has an osmolality of 305 mOsm/kg, thus the inside/outside transmembrane gradient was about 415 mOsm/kg. A 2% sodium chloride solution has an osmolality of about 650 mOsm/kg, yielding an inside/outside liposomal transmembrane gradient of 70 mOsm/kg when the liposomes were placed in 2% sodium chloride. After placing the liposomes in the serum or in the 2% sodium chloride, aliquots were withdrawn and the released drug was separated by the spin filtration method. The sample was analyzed by HPLC for free drug in the external medium. The results are shown in Table 4.

TABLE 4

| | Drug Potency | | | | | |
|---|---|---|---|---|---|---|
| | (mg/mL) | | | | | |
| | | | 0.5 | | | |
| Sample | Total | 0 hours | hours | 1 hour | 2 hours | 5 hours |
| FBS/NaCl | nd* | nd* | — | — | nd* | nd* |
| FBS/liposomes | 0.41 | 0.0007 | — | — | 0.0007 | 0.0013 |
| NaCl/liposomes | 0.41 | 0.0002 | 0.0001 | 0.0001 | 0.0002 | 0.0002 |

*nd = none detected

The amount of drug detected in FBS was about 0.0013 mg/mL after 5 hours incubation. The studies indicated that up to 50 μg drug could bind to the filter, and with this taken into consideration, it is seen that the drug released from liposomes incubated in fetal bovine serum was less than 13% of the total liposomal drug load. Thus, the liposomes had excellent drug retention, even in the presence of a transmembrane osmotic gradient as large as 415 mOsm/kg. Accordingly, a liposome composition having a transmembrane gradient of at least about 200 mosm/kg, preferably of at least about 300 mOsm/kg, still more preferably of about 400 mOsm/kg, and a drug load of greater than about In another study, described in Example 3, liposomes comprised of HSPC/mPEG-DSPE, of HSPC/cholesterol/mPEG-DSPE, or of PHSPC/mPEG-DSPE were prepared using as a hydration medium a solution of 50 w/v % hydroxypropyl-β-cyclodextrin and drug. The liposomes were first diafiltered against a 2% sodium chloride solution (plus 10 mM histidine, pH 6.5). After about half of the formulation was removed, the rest was dialyzed against a 1% sodium chloride solution. The formulations were then sterile filtered using 0.22 μm filter at elevated temperatures (>70° C. for the HSPC-containing formulations and about 45° C. for the PHSPC formulations) and then characterized for particle size, drug concentration, and free drug content post sterile filtration. The results are shown in Table 5.

TABLE 5

| | | Particle | | | | | | |
|---|---|---|---|---|---|---|---|---|
| Liposome | | Size nm | Lipid | Drug | Drug:lipid | Free | | Osmolality |
| Formulation external | Vial | (90/30 | Conc. | Conc. | Ratio | drug¹ | | (mOsm/ |
| buffer | No. | degree) | (mM) | (mg/mL) | (g/mole) | (mg/mL) | pH | kg) |
| HSPC:mPEG | 1 | 171.7/210.3 | 96.2 | 2.79 | 29.0 | 0.08 | 6.8 | 705.0 |
| (95:5) 2% NaCl | 2 | 168.0/204.3 | | | | | 6.82 | 707.3 |
| HSPC:mPEG | 1 | 158.0/213.7 | 91.8 | 2.58 | 28.1 | 0.88 | 6.78 | 413.0 |
| (95:5) 1% NaCl | 2 | 172.78/209.7 | | | | | 6.77 | 412.0 |
| HSPC:Chol:mPEG- | 1 | 189.3/223.3 | 130.9 | 3.03 | 23.1 | 0.05 | 6.89 | 678.0 |
| DSPE (50:45:5) 2% | 2 | 188.7/223.0 | | | | | 6.89 | 675.5 |
| NaCl | | | | | | | | |
| HSPC:Chol:mPEG- | 1 | 187.0/227.6 | 129.2 | 2.88 | 22.3 | 0.65 | 6.84 | 336.0 |
| DSPE (50:45:5) 1% | 2 | 184.3/236.6 | | | | | | |
| NaCl | | | | | | | | |
| PHSPC:mPEG- | 1 | 187.3/249.3 | 91.5 | 3.37 | 36.8 | 0.09 | 6.59 | 712.0 |
| DSPE (95:5) 2% | 2 | 185.3/256.0 | | | | | 6.6 | 716.5 |
| NaCl | | | | | | | | |
| PHSPC:mPEG- | 1 | 186.6/251.6 | 60.0 | 2.00 | 33.3 | 0.38 | 6.64 | 245.0 |
| DSPE (95:5) 1% | 1 | 183.6/251.0 | | | | | | |
| NaCl | | | | | | | | |

¹Free drug was measured post-sterile filtration which was performed at elevated temperatures. The free drug post diafiltration was very low (<0.02 mg/mL).

The data in Table 5 shows, first, that the liposomes have high drug concentrations, between 2.0-3.4 mg/mL of entrapped drug. Second, comparing formulations with the same lipid compositions that were diafiltered against 1% NaCl and against 2% NaCl, it is seen that the drug/lipid ratios are essentially the same (decreased by 4% to 10%), indicating no significant additional drug leakage during the process of diafiltration against 1% NaCl (which was carried out at room temperature), even though there is a significant osmotic gradient across the lipid bilayer membranes. A 50% hydroxy-β-propyl cyclodextrin solution has an osmolality of around 700-720 mOsm/kg (see last column in Table 5). A 2% NaCl solution is essentially isoosmotic with a 50% hydropxy-o-propyl cyclodextrin solution. A 1% NaCl solution has an osmolality of about 350-360 mOsm/kg. Thus, the transmembrane osomotic gradient when the liposomes are diafiltered against a 1% NaCl solution is at least about 350 mOsm/kg. The data showing that the drug/lipid ratios do not change when the liposomes are diafiltered against a 1% NaCl solution indicates that the rigid lipid composition is stable when subjected to the transmembrane gradient of at least about 350 mOsm/kg.

With continuing reference to Table 5, the high free drug content for the formulations diafiltered against 1% NaCl and then sterile filtered at elevated temperatures was the result of high temperature induced drug leakage. As noted above, the oxazolidinone drug RWJ-416457 has poor water solubility of around 20 μg/mL, and if entrapped in liposomes would be expected to have a drug concentration of about 20 μg/mL. The liposomes prepared with the drug in the form of an inclusion complex with cyclodextrin and with a lipopolymer have a drug concentration of 2-3.4 mg/mL, a 100-fold to 170-fold improvement in liposome entrapped drug concentration. As noted above, liposomes with no lipopolymer failed to provide the desired increased drug concentration (see Table 3). Moreover, the lipid formulation that provides a rigid lipid bilayer is able to stably entrap the cyclodextrin/drug complex, even when the liposomes are placed in a medium suitable for intravenous administration, such as physiological saline, that results in a transmembrane osmotic gradient of greater than 200 mOsm/kg, and even of greater than 300 mOsm/kg. Thus, in various embodiments, a liposome composition is provided that has a drug concentration of a poorly water soluble drug of at least about 1.0 mg/mL, more preferably of at least about 2.0 mg/mL, and still more preferably of 3 mg/mL or more, and an inside higher/outside lower transmembrane osmotic gradient of at least 200 mOsm/kg, more preferably of at least 300 mOsm/kg, still more preferably of at least 350 mOsm/kg.

Example 4 describes preparation of liposomes as described above where the liposomes were sized by extrusion to around 100-130 nm, suitable for in vivo intravenous administration. Results are shown in Table 6.

TABLE 6

| | | | | | | | diameter⁴ |
|---|---|---|---|---|---|---|---|
| composition | process | total lipid¹ | Total lipid¹ | drug² | drug/lipid | Encap³ | (nm. |
| (mol/mol) | stage | (mM) | (mg/mL) | (mg/mL) | (mg/mL) | (%) | 90°/30°) |
| HSPC/Chol/mPEG- | post hydration | 80.5 | 60.3 | — | — | — | — |
| DSPE | post extrusion | 80.7 | 60.4 | 10.2 | 0.168 | — | 134/169 |
| (55:45:05) | post diafiltration | 215.4 | 161.2 | 2.2 | 0.014 | 8.1 | 113/127 |
| HSPC/mPEG- | post hydration | 79.6 | 70.9 | — | — | — | — |
| DSPE | post extrusion | 92.8 | 82.6 | 11.1 | 0.135 | — | 130/165 |
| (95:05) | post sterile | 115.1 | 102.5 | 2.4 | 0.023 | 17.4 | 118/127 |
| | filtration | | | | | | |

¹lipid concentration was measured by phosphate assay.
²drug potency was determined by HPLC assay.
³encapsulation efficiency was calculated by the final drug/lipid ratio divided by the drug/lipid ration post hydration
⁴liposome diameter was measured by a submicron particle sizer (Coultier N4M) with detector set at 90° and 30°.

In another study, described in Example 5, four liposome formulations were prepared as described above with a 50% hydroxypropyl-β-cyclodextrin/oxazolidinone drug complex and the lipids HSPC/cholesterol/mPEG-DSPE or HSPC/mPEG-DSPE. After liposome formation, the suspension was extruded to size the liposomes and then diafiltered against 2% sodium chloride solution (2-3 volume exahanges with 2 w/v % NaCl solution and then 4-5 volume exahanges with 1 w/v % NaCl solution) to remove unentrapped cyclodextrin/drug complexes. Liposome particle size, drug concentration, and concentration of free drug in the external suspension medium were measured immediately and then after one month and after two months of storage at 4° C. The results are shown in Table 7.

TABLE 7

| | | Liposome Diameter | | | Potency | | | Free Drug | | |
|---|---|---|---|---|---|---|---|---|---|---|
| Composition | Formulation | (nm) | | | (mg/mL) | | | (μg/mL) | | |
| (mol/mol) | Number/batch | t = 0 | t = 1 mo | t = 2 mo | t = 0 | t = 1 mo | t = 2 mo | t = 0 | t = 1 mo | t = 2 mo |
| HSPC/Chol/mPEG- | 65FF-4D | 189 | 190 | 193 | 3.03 | 3.08 | 3.01 | 0.08^{a} | 0.06^{a} | 0.10^{b} |
| DSPE | | | | | | | | 0.05^{b} | 0.11^{b} | |
| (55:45:5) | 10 | 115 | 117 | 117 | 1.77 | 1.79 | 1.86 | 0.09^{b} | 0.10^{b} | 0.10 |
| HSPC/mPEG- | 65FF-3D | 170 | 167 | 187 | 2.79 | 2.82 | 2.83 | 0.08^{a} | 0.10^{a} | 0.17^{b} |
| DSPE | | | | | | | | | 0.18^{b} | |
| (95:5) | 11 | 126 | 129 | 128 | 1.89 | 1.91 | 1.94 | 0.05^{b} | 0.05^{b} | 0.05 |

^{a}External free drug separated using spin filtration (method a) or ultracentrifugation (method b), which gives a lower free drug content than method b due to membrane absorption. Drug content measured using HPLC.

Table 7 shows that the liposomes have a drug concentration of greater than 1.5 mg/mL, and specifically, concentrations of 3 mg/mL, 1.8 mg/mL, 2.8 mg/mL and 1.9 mg/mL. The lower concentrations for formulation nos. 10 and 11 were likely due to a relatively smaller particle size compared to formulation 65FF-3D and 65FF-4D. The concentrations had essentially no change after one and two months of storage, indicating the rigid liposome lipid bilayer is able to retain the drug despite the high internal osmolality due to the cyclodextrin.

As noted above, the liposome composition can optionally include a lipid-polymer-ligand targeting conjugate. Liposomes with such a conjugate can be prepared by various approaches. One approach involves preparation of lipid vesicles which include an end-functionalized lipid-polymer derivative; that is, a lipid-polymer conjugate where the free polymer end is reactive or “activated” (see, for example, U.S. Pat. Nos. 6,326,353 and 6,132,763). Such an activated conjugate is included in the liposome composition and the activated polymer ends are reacted with a targeting ligand after liposome formation. In another approach, the lipid-polymer-ligand conjugate is included in the lipid composition at the time of liposome formation (see, for example, U.S. Pat. Nos. 6,224,903, 5,620,689). In yet another approach, a micellar solution of the lipid-polymer-ligand conjugate is incubated with a suspension of liposomes and the lipid-polymer-ligand conjugate is inserted into the pre-formed liposomes (see, for example, U.S. Pat. Nos. 6,056,973, 6,316,024).

II. Methods of Use

The composition described herein finds use in treating various conditions and disorders, depending on the drug entrapped in the liposomes. Liposomes with a cytotoxic agent are suitable for treating conditions of cellular proliferation, such as neoplasms. Liposomes with an entrapped antimicrobial agent, such as an oxazolidinone, find use in treating various bacterial and fungal infections, and in particular in treating multi-drug resistant gram positive bacterial infections.

The liposome formulation is typically administered parenterally, with intravenous administration preferred. It will be appreciated that the formulation can include any necessary or desirable pharmaceutical excipient to facilitate delivery.

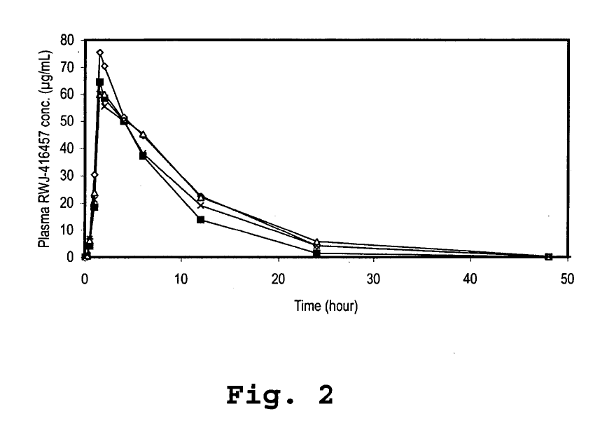

A study was conducted to evaluate the in vivo pharmacokinetics of a liposome composition comprising the drug RWJ-416457 and hydroxylpropyl-β-cyclodextrin. As described in Example 8, liposomes having a lipid composition of HSPC, cholesterol, and mPEG-DSPE (50:45:5 mol %) were prepared using a hydration solution of the oxazxolidinone drug and 45 w/v % hydroxylpropyl-β-cyclodextrin. The formulation was administered intravenously to animals and blood samples were removed at selected times for analysis of plasma drug concentration. Table 8 shows the testing formulation, dosing information, and pharmacokinetic data. FIG. 2 shows the drug concentration in the plasma as a function of time for the four test animals, the time points for each animal represented by a diamond, square, triangle or x symbol.

TABLE 8

| | Formulation | Dose | Drug Conc. | Dose Volume | Cmax | AUC | T_{1/2} |
|---|---|---|---|---|---|---|---|
| Animal No. | Lot No. | (mg/kg) | (mg/mL) | (mL/kg/day) | (μg/mL) | (μg * h/ml) | (h) |
| 1 | FD-17 | 20 | 4.07 | 4.9 | 76 | 697.7 | 4.85 |
| 2 | FD-17 | 20 | 4.07 | 4.9 | 65 | 509.4 | 3.69 |
| 3 | FD-17 | 20 | 4.07 | 4.9 | 60 | 610.1 | 4.94 |
| 4 | FD-12 | 20 | 4.71 | 4.71 | 60 | 704 | 5.29 |

It is to be understood that the determination of the appropriate dose regimen for any given drug in the liposomes and for a given patient is well within the skill of the attending physician. Since the proper dose may vary from person to person based on the age and general state of health, it is a common practice of physicians to “dose-titrate” the patient; that is, to start the patient on a dosing regimen which is at a level below that required to produce the desired response, and gradually increase the dose until the desired effect is achieved. Alternatively, the attending physician can rely on the recommended dose for the given drug when administered in free form.

III. Examples

The following examples further illustrate the invention described herein and are in no way intended to limit the scope of the invention.

Materials: Hydrogenated soy phosphatidylcholine (HSPC) was obtained from Genzyme Corp. (Cambridge Mass.). Partially hydrogenated soy phosphatidylcholine (PHSPC) was obtained commercially. Cholesterol was obtained from Solvay Chemicals, Inc. (Houston Tex.). Hydroxypropyl-β-cyclodextrin (Trappson®) was obtained commercially from Cyclodextrin Technologies Development Inc. (CTD Inc.). Sulfobutylether-β-cyclodextrin (Captisol®) was from Cydex Inc.

### EXAMPLE 1

### Preparation of Liposomes

A. Drug/Cyclodextrin Complex

The solubility of an antimicrobial oxazolidinone drug identified as RWJ-416457 in 40 w/v % hydroxylpropyl-β-cyclodextrin and in 20 w/v % sulfobutylether-β-cyclodextrin under various conditions of pH, temperature, and incubation time was determined as follows. About 15 mgs of drug was added to 5 mL of each cyclodextrin solution followed by incubation under one of three conditions: (1) with stirring at 45° C. for 2 hours; (2) with stirring at 45° C. for 2 hours followed by stirring at room temperature (25° C.) for 3 days; or (3) with stirring at room temperature (25° C.) for 3 days. After incubation, any non-solubilized drug crystals were removed either by centrifugation using a bench top centrifuge at 3000 rpm or by filtration using a hand-held syringe with a filter with 0.2 μm pore size. The drug concentration of the supernatant or the filtrate was then determined by high performance liquid chromatography (HPLC). The results, summarized below, indicate that the drug solubility in 40 w/v % hydroxypropyl-β-cyclodextrin is above 9 mg/mL and is above 4.5 mg/mL in 20% sulfobutylether-β-cyclodextrin.

Solubility of RWJ-416457 in Hydroxypropyl-β-cyclodextrin
and sulfobutylether-β-cyclodextrin

| | Temp Condition | | | | | | | | | | | | | | |
|---|---|---|---|---|---|---|---|---|---|---|---|---|---|---|---|
| | 45° C. 2 hrs | | | | | | | | 45° C. 2 hrs + 3 days RT | | | | 3 days RT | | |
| | Centrifuge | | | | Filter | | | | Filter | | | | Filter | | |
| | Sample No. | | | | | | | | | | | | | | |
| | 2 | 4 | 6 | 7 | 2 | 4 | 6 | 7 | 2 | 4 | 6 | 7 | 1 | 3 | 5 |
| pH | 8.54 | 3.21 | 4.58 | 6.94 | 8.54 | 3.21 | 4.58 | 6.94 | 8.54 | 3.21 | 4.58 | 6.94 | 8.54 | 3.21 | 4.58 |
| HP-β-CD | 40 | | | — | 40 | | | — | 40 | | | — | 40 | | |
| (%) | | | | | | | | | | | | | | | |
| SBE-β-CD | — | | | 20 | — | | | 20 | — | | | 20 | — | | |
| (%) | | | | | | | | | | | | | | | |
| RWJ-416457 | 9.37 | 9.23 | 9.17 | 4.75 | 9.26 | 9.16 | 9.16 | 4.55 | 9.46 | 9.08 | 9.1 | 4.45 | 8.13 | 7.6 | 7.21 |
| (mg/mL) | | | | | | | | | | | | | | | |

B. Preparation of Liposomes Without Polyethylene glycol

A hydration solution with 50% cyclodextrin was prepared by dissolving hydroxypropyl-β-cyclodextrin (100 g), 15 mM NaCl (0.175 g) and 10 mM histidine (0.310 g) in 200 mL buffer (10 mM histidine and 15 mM NaCl) and heating at about 45° C. Four grams (4 g) of an antimicrobial oxazolidinone drug (RWJ-416457) was added to the cyclodextrin solution, warmed to about 45-50° C. for about 2 hours. The pH was adjusted to 6.5. The drug/cyclodextrin solution was allowed to sit overnight and was then filtered through a 0.2 μm filtering system to remove undissolved drug.

A similar solution containing 30% cyclodextrin was prepared by dissolving 30 g of hydroxypropyl-β-cyclodextrin in a 100 mL of water. The solution also contained 10 mM histidine and 15 mM NaCl. The final osmolality was 451 mOsm/kg (Wescor Osmometer) and the pH was 6.8. Drug (RWJ-425457) was added to the cyclodextrin solution, incubated (45° C. for 2 hours), and filtered, to yield a drug concentration of 9.9 mg/mL.

A similar solution containing 20% cyclodextrin was prepared by dissolving 20 g of hydroxypropyl-β-cyclodextrin in a 100 mL of buffer (10 mM histidine and 15 mM NaCl). The final osmolality was 199 mOsm/kg (Wescor Osmometer) and the pH was 6.8. Drug (RWJ-425457) was added to the cyclodextrin solution, incubated (45° C. for 2 hours) and filtered, to yield a drug concentration of 4.8 mg/mL.

Lipid solutions comprised of HSPC (7.1 g) or of HSPC:cholesterol (55:45 mol/mol; 3.9 g HSPC, 1.6 g cholesterol) were prepared by adding anhydrous ethanol (10 mL). Placebo liposomes were prepared by adding to the lipid solutions 90 mL warm saline (60-65° C.). Active drug-containing liposomes were prepared by adding to the lipid solution 90 mL of the 50% cyclodextrin/drug solution, warmed to 60-65° C. The solutions were stirred for one hour. Similarly lipid suspensions with the 30% cyclodextrin/drug solution or the 20% cyclodextrin/drug solution were prepared.

Six different liposome formulations with no PEG were prepared, identified in the table below and in Table 3 as formulation nos. 1-6:

| Formulation | Lipid Components | | Osmolarity |
|---|---|---|---|
| No. | (mol/mol) | Hydration Medium | (mOsmo) |
| 1 (control) | HSPC | saline/EtOH (10 v/v %) | n/a |
| 2 (control) | HSPC:chol (55:45) | saline/EtOH (10 v/v %) | n/a |
| 3 | HSPC | drug (12.95 mg/mL)/HPβCD (50 w/v %)/EtOH (10 | 720 |
| | | v/v %) | |
| 4 | HSPC:chol (55:45) | drug (12.95 mg/mL)/HPβCD (50 w/v %)/EtOH (10 | 720 |
| | | v/v %) | |
| 5 | HSPC:chol (55:45) | drug (9.9 mg/mL)/HPβCD (30 w/v %)/EtOH (10 v/v %) | 451 |
| 6 | HSPC:chol (55:45) | drug (4.8 mg/mL)/HPβCD (20 w/v %)/EtOH (10 v/v %) | 198 |

C. Preparation of Lirosomes with Polyethylene GIYcoI

Liposomes were prepared with 1 mole percent, 2.5 mole percent, and 5 mole percent mPEG-DSPE. The procedure for preparation of liposomes having 5 mole percent mPEG-DSPE is detailed, and the formulations with 1 mole percent and 2.5 mole percent mPEG-DSPE were prepared similarly with adjustments in the total lipids for the differing amount of mPEG-DSPE.

A lipid mixture of HSPC/chol/mPEG-DSPE 50:45:5 mol/mol (1.74 g HSPC, 0.615 g cholesterol, and 0.56 g mPEG-DSPE) was solubilized in 4.0 mL ethanol at about 55-65° C. A 50% hydroxypropyl-β-cyclodextrin solution was made by solubilizing 20 g of the cyclodextrin in a total volume of 50 mL water. In a separate container 0.2 g of drug (RWJ416457) was placed in 40 mL of the 50 w/v % cyclodextrin solution to solubilize the drug. The lipid solution was hydrated by mixing with 40 mL of one of the drug/cyclodextrin solutions at 56-58° C. with stirring for 1 hour to form a suspension of liposomes.

The formulations prepared with the 50 w/v % cyclodextrin/drug hydration media and the lipid mixture are summarized in the table below and in Table 3 as formulation nos. 7, 9, and 10. Formulation no. 8 was prepared similarly, except that a hydration medium with 20 w/v % cyclodextrin/drug was used.

A liposome formulation with no cholesterol, formulation no. 11, was also prepared as follows. A lipid mixture of HSPC/mPEG-DSPE 95:5 mol/mol (11.3 g HSPC and 2.1 g mPEG-DSPE) was solubilized in 10 mL ethanol at about 65 ° C. A 50% hydroxypropyl-β-cyclodextrin solution was made by solubilizing 50 g of the cyclodextrin in a total volume of 100 mL water. The osmolality was 720 mOsm/kg and the pH was 7.4. In a separate container 1.004 g of drug (RWJ416457) was placed in 100 mL of the 50 w/v % cyclodextrin solution. The mixture was stirred for 1 hour at 45° C. and then 3 hours at room temperature followed by filtration to removw any un-dissolved drug in the solution. The final drug concentration assayed by HPLC was 7.58 mg/mL and the osmolality of the hydration medium was 771 mOsm/kg. The lipid solution was then mixed with 90 mL of the drug/cyclodextrin solution (pre-warmed to 65° C.) followed by stirring at 65° C. for 1 hour to form a suspension of liposomes, identified as formulation no. 11.

| Formulation | Lipid Components | | Osmolarity |
|---|---|---|---|
| No. | (mol/mol) | Hydration Medium | (mOsmo) |
| 7 | HSPC/chol/mPEG- | drug (12.6 mg/mL)/HPβCD (50 w/v %)/EtOH (10 v/v %) | 720 |
| | DSPE | | |
| | (52.5:45:2.5) | | |
| 8 | HSPC/chol/mPEG- | drug (5.1)/HPβCD (20 w/v %)/EtOH (10 v/v %) | 198 |
| | DSPE | | |
| | (50:45:5) | | |
| 9 | HSPC/chol/mPEG- | drug (13 mg/mL)/HPβCD (50 w/v %)/EtOH (10 v/v %) | 720 |
| | DSPE | | |
| | (54:45:1) | | |
| 10 | HSPC:chol:mPEG- | drug (13.0 mg/mL)/HPβCD (50 w/v %)/EtOH (10 v/v %) | 720 |
| | DSPE (50:45:5) | | |
| 11 | HSPC/mPEG- | drug (13.0 mg/mL)/HPβCD (50 w/v %)/EtOH (10 v/v %) | 720 |
| | DSPE | | |
| | (95:5) | | |

D. Analysis of Liposome Compositions

For liposome formulation nos. 1-4 and 7-9, the liposome compositions were sized by sequential extrusion through 0.4 μm (2-4 passes), 0.2 μm (4-5 passes), and 0.1 μm (2 passes for formulation nos. 1-9, and 3-5 passes for formulation nos. 10 and 11) polycarbonate filters. Liposome particle size was measured by dynamic light scattering (Coulter, N4MD) and the results are shown in Table 3.

The liposome formulations containing drug and 50w/v % HP,CD (formulation nos. 3, 4, 5, 7, 9, and 10) were diafiltered to remove un-entrapped drug/cyclodextrin using a 300k molecular weight cut-off ultrafiltration cartridge with 2% NaCl and 10 nM histidine (osmolality 650 mOsm/kg, pH 6.7). The diafiltered solution was concentrated from about 100 mL to about 54-66 mL. Liposome particle sizes, as measured using dynamic light scattering in two of the formulations, identified as formulation no. 3 and formulation no. 4, were determined. Formulation no. 3 had a particle size post diafiltration of about 643-684 nm (300/900 measurements). Liposome size in formulation no. 4 was about 189-290 nm (30°/90° measurements) post diafiltration.

For liposome formulation nos. 5-6, the mixture was extruded in a Lipex extruder through 0.4 μm polycarbonate membrane for 4 times, 0.2 μm polycarbonate membrane 5 times, and 0.1 μm polycarbonate membrane for 2 times at 65° C. The extruded liposome was then diafiltered against 1% NaCl (formulation no. 5) or 0.6% NaCl (formulation no. 6) with 10 mM histidine, pH ˜6.8, using A/G ultrafiltration cartridge (300k MWCO, area 110 cm²) for 6 volume exchanges. The formulation was concentrated down to the half of the original volume. After diafiltration of formulation no. 6, the formulation was concentrated from 82 mL to 54 mL. The formulations were characterized for particle size, drug potency, and free drug content:

| | | | | | | | Estimated |
|---|---|---|---|---|---|---|---|
| Formulation | | Particle Size (nm) | | | Free Drug | Osm | Final Lipid |
| No. | In-Process | 90 deg | 30 deg | Potency (mg/ml) | (mg/ml) | (mOsm/kg) | Conc. (mM) |
| 5 | Hydration | | | 9.90 | | | |
| | Extrusion | 217 | 488 | | | | |
| | Pre-DF | 232 | 417 | | | | |
| | Post DF | 204 | 462 | 0.22 | 0.04 | 320 | 146.6 |

| Formulation | | Particle Size (nm) | | | Free Drug | Osm | Lipid Conc. |
|---|---|---|---|---|---|---|---|
| No. | In-Process | 90 deg | 30 deg | Potency (mg/ml) | (mg/ml) | (mOsm/kg) | (mM) |
| 6 | Hydration | | | 4.80 | | 198 | 90 |
| | Extrusion | 220 | 438 | | | | |
| | Pre-DF | 197 | 199 | | | | |
| | Post DF | 191 | 282 | 0.17 | 0.07 | 188 | 80.2 |

After diafiltration, drug potency of all liposome formulations was determined using HPLC. The results are shown in Table 3.

### EXAMPLE 2

### Drug Leakage Study

A lipid mixture of HSPC/mPEG-DSPE 95:5 mol/mol (11.26 g HSPC from Lipoid and 2.1 g mPEG-DSPE) was solubilized in 10 mL ethanol at about 65° C.

A 50% hydroxypropyl-β-cyclodextrin solution was made by solubilizing 50.0 g of the cyclodextrin in a total volume of 100 mL water. The final osmolality was 720 mOsm/kg (Wescor Osmometer) and the pH was 7.4. In a separate container 1.004 g of drug (RWJ416457) was placed in 100 mL of the 50 w/v % cyclodextrin solution. The mixture was stirred for 4 hours at room temperature followed by filtration to remove any drug in the external solution. The final drug concentration assayed by HPLC was 7.58 mg/mL and osmolality was 771 mOsm/kg.

The lipid solution was then mixed with 90 mL of the drug/cyclodextrin solution (prewarmed to 65° C.) followed by stirring at 65° C. for 1 hour to form a suspension of liposomes.

The lipid suspension was extruded at 62° C. using a Lipex extruder (Lipex Inc.) with 4 passes with a 0.4 μm filter and 6 passes with a 0.2 μm filter (polycarbonate membranes). 10 mL of saline was added after the 0.4 μm filtration step to reduce the viscosity. The liposome mean diameter, measured by dynamic light scattering, was approximately 250 nm.

External cyclodextrin and drug were then removed by diafiltration using a cartridge (AG Tech. UFP-300-E, NMWC 300K, 110 cm² area) against a 2% NaCl solution (osmolality =653 mOsm/kg, pH=6.74). Eight volume exchanges were performed to ensure complete removal of external drug and cyclodextrin. The formulation was finally concentrated to about 65 mL using the diafiltration setup. The final drug potency was 1.94 mg/mL assayed by HPLC and the drug concentration in the permeate of the last exchange was 0.01 mg/mL, indicating that more than 99.8% of external drug has been removed and the final sample contained 99.5% of the drug inside the liposomes.

One part of the liposome formulation was mixed with four parts of fetal bovine serum (FBS; Sigma) or with 2% NaCl solution as a control and incubated at 37° C. for up to 5 hours. A 1 mL aliquot was then subjected to spin filtration (VIVASPIN 20 mL CONCENTRATOR with 300k MWCO spun for 60 min at 3700 rpm at room temperature). About 200-500 μL of permeate was collected containing the drug released from the liposomes and analyzed for concentration by HPLC. The results are shown in Table 4. A study was conducted to determine the amount of drug adsorption onto the filter membrane by subjecting an equal volume (1 mL) of hydroxypropyl-β-cyclodextrin containing 0.41 mg/mL (equivalent to the drug concentration in the active liposome samples) to the same spin filtration process. The result indicated that approximately 40-50 μg drug was absorbed onto one filter. Thus, the drug released from liposomes incubated in 80% FBS was no more than 50 μg, which corresponds to less than 13% of the total liposomal drug load.

### EXAMPLE 3

### Liposome Preparation

Liposomes were prepared according to the procedure described in Example 2. Three different lipid compositions were used, 95:5 mol/mol fully hydrogenated soy phosphatidylcholine (HSPC):methoxypolyethylene glycol-distearoylphosphatidylethanolamine (mPEG-DSPE); 50:45:5 mol/mol HSPC: cholesterol:mPEG-DSPE; and 95:5 mol/mol partially hydrogenated soy phosphatidylcholine (PHSPC):mPEG-DSPE. The initial lipid concentration at the hydration stage was 100 mM. The lipid mixtures were hydrated at 65° C. for 1 hour with the oxazolidinone drug (RWJ-416457) in 50% hydroxylpropyl-β-cyclodextrin, 10 mM histidine, pH 6.5. The liposomes were extruded at 65° C. in a Lipex extruder using four passes with a 0.4 μm membrane, and either 6 or 8 passes with a 0.2 μm membrane. The extruded formulations were diafiltered by exchanging against buffer containing 2% NaCl, which is in isoosmotic balance with the internal 50% hydroxylpropyl-β-cyclodextrin, for 8 volume exchanges.

Then, for the liposome compositions containing HSPC, about half of each liposome formulation was removed from the diafiltration system and the remaining amounts were further exchanged with buffer containing 1% NaCl. For the liposome composition containing PHSPC, a 1% NaCl sample was obtained by 1:1 dilution of an aliquot of the formulation balanced with 2% NaCl with 10 mM histidine solution containing no NaCl. The formulations were then subjected to sterile filtration using a 0.22 μm filter at above 70° C. (sterile filtration cartridge temperature was controlled by a circulating water-bath set at about 80° C.) for the HSPC formulation and 45° C. for the PHSPC formulation. The six formulations were characterized for particle size, drug potency (concentration), and free drug content and other parameters, shown in Table 5.

### EXAMPLE 4

### Liposome Preparation

Liposomes comprised of HSPC/cholesterol/mPEG-DSPE (55:45:5) and of HSPC/mPEG-DSPE (95:5) were prepared as described in Example 2, with the following changes. The liposome formulations were extruded additionally through a 0.1 μm filter for 3-5 passes to achieve a liposome size close to 100 nm. Diafiltration was performed by three volume exchanges with 2% NaCl (and 10 mM histidine at about pH 7) to remove ethanol and external drug/hydroxypropyl-β-cyclodextrin, followed by an additional five volume exchanges with isotonic buffered NaCl solution (1% NaCl, 10 mM histidine pH=7). The formulations were concentrated using the diafiltration setup to increase the final drug concentration. The liposomes were characterized and the results are shown in Table 6.

### EXAMPLE 5

### Stability of Liposomes Containing RWJ-416457/cyclodextrin

Liposomes comprising the drug RWJ-416457 and 50 w/v % hydroxylpropyl-β-cyclodextrin were prepared as described in Example 1. The mean liposome diameter, drug concentration (potency), and free drug concentration in the external suspension medium were measured after preparation. The formulations were stored at 4° C. and the parameters were tested again after 1 month and 2 months. The results are shown in Table 7.

EXAMPLE 6

### In vivo Pharmacokinetics of Liposomes Containing RWJ-41 6457/cyclodextrin

Liposomes comprising the drug RWJ-416457 and 45 w/v % hydroxylpropyl-β-cyclodextrin were prepared as described in Example 1. The lipid composition was HSPC/CHOL/mPEG-DSPE (50:45:5 mol %). Two lots of liposomes were formed, and the final drug potency of each lot was 4.07 mg/mL and 4.71 mg/mL.

Male purebred beagles (8-13 kg) were fitted with an indwelling percutaneous catheter in a jugular vein for dosing by intravenous infusion. Following catheterization, the dogs were fitted with an infusion jacket and collar. The catheter of each dog was flushed with heparinized saline in order to maintain the patency of the catheter. Dose volumes were based on the most recently recorded body weight.

An infusion at a rate of 3 mL/hr (50 μL/min) was initiated, and if no adverse reactions were observed, the infusion rate was increased after 15 minutes to 30 mL/hr (500 μL/min) until the target dose volume was delivered. Blood (2 mL) was collected from the non-catheterized jugular vein or an appropriate peripheral vein at each time point. Tripotassium ethylenediaminetetraacetic acid (K3-EDTA) was used as the anticoagulant. Blood samples were chilled and then centrifuged within 1 hour of collection. The plasma was harvested and frozen on dry ice until analysis.

Table 8 shows the testing formulation, dosing information, and summary of the results. FIG. 2 shows the drug concentration in the plasma as a function of time for the four test animals.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A composition, comprising
liposomes comprised of a vesicle-forming lipid having a phase transition above about 40° C. and of between about 1-20 mole percent of a lipid derivatized with a hydrophilic polymer;
entrapped in said liposomes, a complex comprised of a hydrophobic drug and a cyclodextrin compound, said cyclodextrin present in a concentration above about 100 mg/mL.

2. The composition of claim 1, wherein said vesicle-forming lipid is a saturated phosphatidylcholine.

3. The composition of claim 2, wherein said saturated phosphatidylcholine is distearolyphosphatidylcholine or hydrogenated soy phosphatidylcholine.

4. The composition of claim 1, wherein said liposome are further comprised of cholesterol.

5. The composition of claim 1, wherein said cyclodextrin is selected from the group consisting of methylated, phosphated, sulfated, sulfoalkyl ether, carboxymethyl, and succinylated cyclodextrins.

6. The composition of claim 5, wherein said cyclodextrin is selected from sulfobutyl ether β-cyclodextrin or hydroxyl propyl β-cyclodextrin.

7. The composition of claim 1, wherein said cyclodextrin is present at a concentration of greater than 200 mg/mL.

8. The composition of claim 1, wherein said cyclodextrin is present at a concentration of greater than 300 mg/mL.

9. The composition of claim 1, wherein said cyclodextrin is present at a concentration of greater than 400 mg/mL.

10. The composition of claim 1, wherein said cyclodextrin is present at a concentration of greater than 500 mg/mL.

11. The composition of claim 1, wherein said liposomes further comprise, in liposome entrapped form, a water soluble polymer, a salt, or both.

12. The composition of claim 11, wherein said water soluble polymer is selected from the group consisting of hydroxypropyl methylcellulose, polyvinyl pyrrolidone, and gelatin.

13. The composition of claim 11, wherein said salt is selected from the group consisting of sodium chloride, sodium acetate, sodium citrate, sodium salicylate, and sodium benzalkonium.

14. A liposome composition prepared according to a process comprising;
providing lipids comprised of a vesicle-forming lipid having a phase transition above about 40° C. and of between about 1-20 mole percent of a lipid derivatized with a hydrophilic polymer,
combining the lipids with a drug-cyclodextrin solution to form liposomes having a concentration of cyclodextrin of greater than about 100 mg/mL;
processing said liposomes to obtain a desired particle size.
